(19) United States
(12) Patent Application Publication
(10) Pub. No.: US 2013/0156812 A1
(43) Pub. Date: Jun. 20, 2013

(54) Composition of Antigen and Method of Sublingual Administration

(71) Applicants: Stephen Hauer, Highland Park, IL (US); Kristin Holm, Johnston, IA (US)

(72) Inventors: Stephen Hauer, Highland Park, IL (US); Kristin Holm, Johnston, IA (US)

(21) Appl. No.: 13/433,117

(22) Filed: Mar. 28, 2012

Related U.S. Application Data
(60) Provisional application No. 61/554,969, filed on Nov. 2, 2011.

(51) Int. Cl. A61K 9/00

(52) U.S. Cl. CPC A61K 9/0056
USPC 424/275.1

(57) ABSTRACT

A method of sublingually administering an effective amount of an antigenic composition to a mammal in order to produce a mucosal and systemic immune response.

### BACKGROUND OF THE INVENTION

Allergic disease is an increasingly prevalent health problem in humans as well as other mammals such as household pets. Allergic disease is characterized by immunological reaction to foreign, non-pathologic substances, also known as allergens. Allergic disease is specific in that an individual sensitized to particular allergens will not necessarily exhibit an allergic reaction to other substances known to trigger allergic disease. Immune systems of individuals sensitized to particular allergens will contain allergen-specific IgE antibodies and allergen-specific T-cells.

Allergic disease is characterized by an inappropriate immunological reaction to foreign non-pathogenic substances. Important clinical manifestations of allergy include asthma, hay fever, eczema, and gastro intestinal disorders. Allergic reactions are prompt and peak within 20 minutes upon contact with the offending allergen. Furthermore, allergic reactions are specific in the sense that a particular individual is sensitized to particular allergen(s), whereas said individual will not necessarily exhibit an allergic reaction to other substances known to cause allergic disease.

Allergens can present themselves as environmental sources in the air. These environmental sources are remarkably universal, are responsible for the vast majority of all allergies, and include pollens, mold spores, micro-fungi, animal dander, insect and house dust mite faecal particles. Together, these sources are responsible for approximately 75% of all allergies. In addition to sensitivity to environmental sources, allergic disease also commonly presents itself as sensitivity to specific foods.

Allergic disease management comprises diagnosis and treatment, including prophylactic treatments. Allergic disease is commonly diagnosed by the presence of allergen specific IgE antibodies, which leads to identification of a specific allergen source. In many cases a careful anamnesis may be sufficient for the diagnosis of allergy and for the identification of the offending allergen source material. Most often, however, the diagnosis is supported by more intrusive, objective measures, such as skin prick test, blood test, or provocation test.

Therapeutic options for treating allergic disease fall into three major categories. The first option is allergen avoidance, which may be tenable with regards to food sensitivities but generally lacks practicality or economic feasibility with regards to environmental, air-borne allergens such as household dust mites or pollens.

The second, widely used option involves the use of symptomatic drugs such as anti-histamines and steroids. Symptomatic drugs are safe and efficient; however, like avoidance, they fail to alter the course of the progressive disease, they fail to prevent future allergic responses, and in their application to non-humans such as household pets, they remain exceedingly difficult, and therefore undesirable.

The third therapeutic option is allergen-specific immunotherapy, which employs the immune system's “memory” for previously encountered antigens such that a second, more vigorous response is elicited upon successive exposures to the same antigen.

In conventional medicine, the adaptive immune response of mammals is leveraged in vaccines, which are routinely administered for diseases such as measles or influenza. Specifically, denatured antigens are administered to a mammal. While generally harmless to said mammal, the denatured antigens activate the adaptive immune system to produce antibodies, which can neutralize proteins similar to the denatured antigens given. Later, when the body encounters a virus or other antigen having a similar make-up, the body can effectively produce antibodies against the invader due to the adaptive immune system.

Generally, immunotherapy against infectious agents as described can be performed efficiently using only a single or a few high-dose immunizations, also known as vaccinations. This is possible because vaccination treatments to prevent specific diseases are administered when the infectious agents are not already present in a patient. This specific treatment strategy is not effective for patients suffering from allergic disease because a pathological immune response is already ongoing in the form of allergen-specific IgE antibodies and T-cells.

Therefore, allergen-specific immunotherapy must modify allergic immune responses that are already taking place. To accomplish this, desensitization immunotherapy has been used for the treatment of allergic disease since the beginning of the past century. Desensitization immunotherapy involves exposing a patient to successive, increasing doses of an allergen to elicit an immune response. Over the course of immunotherapy, the immune system adapts to the presence of a specific allergen, which confers future immunity against said allergen. By first identifying the IgE antibodies already present in a patient, immunotherapy treatment is tailored to address the specific set of allergens to which an individual patient exhibits sensitivities.

In spite of its virtues, allergen-specific immunotherapy is not in widespread use, primarily for two reasons. One reason is inconvenience. Conventional allergy-specific immunotherapy is carried out in a specialist's office using multiple subcutaneous immunizations applied over an extended time period, generally 2-3 years. The other, more important reason is the risk of allergic side reactions. Further, following each injection a patient must remain under medical supervision for at least thirty minutes due to the risk of anaphylactic side reactions, which, though rare, are potentially life-threatening.

While subcutaneous allergen injection remains the most widely used method in desensitization immunotherapy efforts, an increasingly popular and clinically substantiated treatment associated with a high level of patient compliance is sublingual administration. Sublingually administered immunotherapy minimizes the risk of the allergic side reactions inherent in subcutaneous injection thereby enabling self-administration at home. Regimented sublingual allergen administration is also superior to intermittent injections at maintaining adequate blood concentrations. For non-human patients, such as household pets, sublingual administration provides a far easier alternative to difficult, owner-administered injections or less effective introduction through oral consumption. This provides, among others, the dual benefits of increasing owners' likelihood of maintaining a treatment regiment and facilitating pet compliance via a minimally invasive method of treatment introduction.

### SUMMARY OF THE INVENTION

The present invention takes advantage of the efficacy and ease of administration of the sublingual route as a pathway for introducing allergens into the body for use in allergen-specific immunotherapy treatment of allergic disease. Effective introduction of desensitization therapy treatments to animals, however, requires a mechanism for ensuring that allergenic proteins remain in contract with said animal's sublingual mucosal region for an extended period of time. As prolonged sublingual exposure is a requirement for effective treatment, and animals will not generally willingly allow an administered dose to remain in their mouth or under the tongue long enough for absorption of a therapeutic dose, there remains a problem for effective sublingual immunotherapy administration to non-humans. The present invention solves this problem by allowing pet owners to administer antigenic doses in such a way that the subject pet will enthusiastically comply with its treatment. An added benefit is that this invention provides a method of introduction that increases the likelihood of continued owner compliance as well.

While sublingual introduction is a well-known pathway for use in exposing human patients to allergenic compounds in the course of desensitization therapy, we understand that we are the first to provide a uniquely convenient method tailored for animal compliance over a course of treatment. Whereas human patients voluntarily participating in a course of immunotherapy reliably ensure that antigenic proteins will remain under the tongue for a sufficient amount of time to be absorbed into the bloodstream. In contrast, animals, unaware that they are receiving an allergy treatment, simply spit out or swallow orally administered treatment doses. Antigenic proteins are thereby either lost or destroyed in the gastric and intestinal tracts resulting in little or no bio-availability absorption. Our method, which features minimal difficulty of administration, maximizes exposure of antigens to the mucosal region of an animal's mouth; which, therefore, derives maximum bio-availability and immunological benefits.

In broad terms, the present invention mixes doses of allergenic extracts of multiple well-known environmental and food allergens into a viscous paste appropriately flavored to appeal to subject household pets. In administration of the desensitization treatment, a pet owner spreads the paste-allergen mixture on a plate or the bottom of a pet's food bowl. The viscosity of the treatment paste-allergen mixture is tailored to necessitate that a pet must lick the mixture numerous times, over the course of several minutes for full consumption. A pet willingly does so due to the appealing flavoring of the mixture. As a pet repeatedly licks at the paste-allergen mixture, each successive lick allows a partial dose of the allergen extract to be drawn into the mouth, where with each lick the mouth becomes awash with saliva and the allergen extracts, which thereby come into contact with the sublingual mucosal membrane. The cumulative dosage and duration that the allergenic proteins remain in contact with the sublingual mucosal membrane provides for an effective course of treatment.

Allergen-specific immunotherapy is generally tailored to the specific allergy profile of individual human patients via skin or blood testing and analysis. Such specification is possible in animal subjects as well. However, such testing is expensive, there are few specialists in veterinary dermatology, a small subset of all doctors of veterinary medicine, who are able to perform this procedure and testing requires multiple veterinary visits. To avoid this expensive, invasive and time-consuming testing, it is possible to begin a course of treatment using allergenic doses of known environmental and food allergy sources. Such doses are of a concentration below that which triggers an allergic reaction, but still provide a clinically relevant universal course of treatment appropriate for any animal patient.

Though repetition of a single, set dosage can be used throughout the treatment protocol, increasing dosages over time is also possible if deemed therapeutically necessary. This can be accomplished by simply incrementally increasing the amount of the antigen-paste mixture provided to an animal patient.

It is well known in the art that the efficacy of any sublingual therapeutic treatment requires that an antigenic compound must maintain contact with the sublingual mucosal membrane. Said contact must continue for a sufficient duration to permit passage of a therapeutic dosage of an antigenic compound through the sublingual mucosal membrane and into the circulatory system of a mammalian patient.

Required contact duration varies by specific antigenic compound, however, failure to maintain contact between antigenic compound and sublingual mucosal membrane for sufficient duration significantly impedes immunological goals. Furthermore, should an antigenic compound be ingested rather than absorbed, said ingestion and subsequent gastro-ontological passage renders it clinically ineffective.

Whereas a high degree of compliance is possible in humans receiving therapeutic compounds via sublingual mucosal introduction, a problem is presented in ensuring patient compliance in non-human mammalian patients. It is the understanding of the inventors that several methods are currently in use to attempt sublingual mucosal introduction of therapeutic compounds in non-human mammalian subjects. One such method is a sprayed mist directed into the patient animal's mouth. While this method has exhibited some success for single-use dosages, such as vaccinations, its usefulness decreases when applied to therapeutic treatments requiring repeated administrations, especially those that also require extended bio-availability duration. Such is the case for desensitization therapies.

This invention addresses a need in the art for a method of sublingual mucosal introduction in non-human subjects that facilitates an ease of administration over a prolonged course of therapeutic treatment while also allowing extended duration of contact with the mucosal membrane.

### DETAILED DESCRIPTION

Various further preferred features and embodiments of the present invention will now be described through non-limiting examples. It will be apparent to one of ordinary skill in the art that the present invention may be practiced without the following specifics.

In a preferred embodiment, a pet-owner administrator spreads a predetermined quantity of a paste-allergen extract mixture onto the bottom of an animal's food bowl or plate. The paste, which comprises the base of the antigenic mixture, has a viscosity such that an animal must repeatedly lick the paste-allergen mixture in order to achieve full consumption of the entire quantity. With every lick, a small amount of allergen is drawn into the mouth.

In this embodiment, each successive lick delivers a small quantity of allergenic extract into the subject animal's mouth, which will become awash with the allergen mixture. Upon introduction into an animal's mouth by licking, the allergenic extract comes into contact with the sublingual mucosal membrane. The cumulative effect of the subject animal consuming an entire therapeutic dose via licking is the allergenic extract maintaining contact with the sublingual mucosal membrane for a sufficient duration to ensure the desired immunological results.

While a panaceatic combination of allergenic extracts representing all known environmental and food allergens is not feasible, a preferred embodiment of this invention utilizes multiple allergenic extracts. Potential allergenic extracts include, but are not limited to those listed in Table 1. Additional potentially therapeutic allergenic extracts are well known to one of ordinary skill in the art.

TABLE 1
GRASSES

| | |
|---|---|
| Bermuda | Cynodon dactylon |
| Bahia | Paspalum notatum |
| Timothy | Phleum pratense |
| Rye | Lolium perenne |
| Fescue, Meadow | Festuca elatior |
| Johnson | Sorghum halepense |

WEEDS

| | |
|---|---|
| Dock | Rumex patientia |
| Groundsel | Senecio vulgaris |
| Sheep Sorrel | Rumex acetosella |
| Wormwood | Artemisia absinthium |
| Rough Marsh Elder | Iva ciliata |
| Pigweed | Amaranthus palmeri |
| Firebush | Kochia scoparia |
| Lamb's Quarters | Chenopodium album |
| English Plantain | Plantago lanceolata |
| Ragweed | Ambrosia psilostachya |
| Sagebrush | Artemisia tridentata |
| Wingscale | Atriplex canescens |
| Russian Thistle | Salsola kali |
| Cocklebur | Asteraceae xanthium |

FOODS
Beef
Chicken
Corn
Wheat
Beet
Cod Fish
TREES

| | |
|---|---|
| Box Elder | Acer negundo |
| Cypress | Cupressus arizonica |
| Eucalyptus | Eucalyptus obliqua |
| Mulberry | Morus spp. |
| Olive | Olea Europea |
| Elm | Ulmus spp. |
| Hickory | Carya spp. |
| Alder | Alnus spp. |
| Ash | Fraxinus spp. |
| Red Cedar | Juniperus virginiana |
| Maple | Acer spp. |
| Oak | Quercus spp. |
| Privet | Ligustrum vulgare |
| Willow | Salix spp. |
| Pecan | Carya illinoesis |
| Queen Palm | Cocos plumosa |
| Sweet Gum | Liquidambar styraciflua |
| Birch | Betulaceae spp. |
| Cottonwood | Populus deltoides |

MITES

| | |
|---|---|
| House Dust Mite | D. farinae |
| House Dust Mite | D. pteronyssinus |

MOLDS
Alternaria T.
Aspergillus Fumigatus
Cladosporium H.
Penicillium N.

In a preferred embodiment, all allergenic extract materials used in the antigenic composition are originally obtained in a lyophilized form. In order for these allergenic extracts to achieve their optimal antigenic effect, they must be reconstituted prior to their mixture into the viscous paste, which forms the basis for the invention herein disclosed.

Reconstitution is achieved by the introduction of diluents. Said diluents can be comprised solely of glycerin or of a combination of glycerin and saline in a proportion apparent to one of ordinary skill in the art. In a preferred embodiment, however, the reconstitution employs diluents comprised of 50% glycerin and 50% saline by volume and said diluents are introduced to an equal volume of lyophilized allergenic extract.

In an additional embodiment, it is possible to introduce an adjuvant to enhance the extract migration through the mucosal membrane and into a patient mammal's bloodstream.

In preferred embodiments, the present invention includes a viscous paste in which a selection of allergenic extracts of well-known allergens, such as those contained within Table 1, are mixed. The paste is flavored to appeal to an animal undergoing the immunotherapy treatment so that said animal, when presented with the paste-allergen mixture, willingly and repeatedly licks it until the paste-allergen mixture is fully consumed. Said paste also must not react with, impair, or degrade the immunological potency of embedded allergenic extracts.

In addition to the above-mentioned properties, in preferred embodiments the paste also must be of a sufficient viscosity and stickiness such that when spread onto the bottom of a feeding bowl or plate, a pet will require between roughly 2 and 5 minutes for full consumption via repeated licking. The preferred consistency of said past-allergen mixture will be such that one of ordinary skill in the art can prepare and administer the proper dosage without undue difficulty and with common household tools.

Furthermore, in a preferred embodiment, the paste is of a robust physical and chemical nature such spoliation or degradation of embedded immunological allergenic extracts is limited, even through long-term storage at room temperatures.

In a preferred embodiment, the paste is peanut butter. In yet another embodiment, the paste is an organic peanut butter. In a further embodiment, the paste is substituted for a suitably viscous gel.

In another embodiment, the allergen-paste mixture includes additional nutritional enhancements as long as said enhancements do not adversely affect the immunological potency of the embedded allergenic extracts. One such example of a useful nutritional enhancement to the paste-allergen mixture is Omega-3 fatty acid in a concentration that would be apparent to an individual possessing ordinary skill in the art. Other useful nutritional enhancements and proper dosage amounts will be apparent to one of ordinary skill in the art.

In another embodiment, the allergen-paste mixture is supplemented with an agent designed to enhance digestive health and bolster the innate immune system, based in the digestive tract. In a preferred embodiment, one such enhancing agent can be a prebiotic such as fermented yeast. Other suitable prebiotic supplements, and their proper dosage amounts, will be apparent to one of ordinary skill in the art.

In yet another embodiment, the paste-allergen mixture is supplemented with a probiotic agent to enhance digestive health. In a preferred embodiment, the probiotic agent is Bacillus Coagulans GBI-30, 6086, chosen for its minimal vulnerability to temperature variation, pressure fluctuation and shelf-life duration. In other embodiments, the probiotic is lactobacillus, acidophilus, bifidobacteria or another beneficial probiotic apparent to one of ordinary skill in the art.

Upon selection and reconstitution of the multiple allergenic extracts, the extracts are mixed into the viscous paste that comprised the base of the antigenic composition. The amount of each environmental and food allergenic extract to be mixed into the viscous paste is based on product weight ratios and will be apparent to one of ordinary skill in the art. In a preferred embodiment, the allergen extracts are mixed into the viscous paste prior to the addition of supplemental additives such as prebiotic or probiotic agents.

What is claimed is:

1. A method for the sublingual administration of a therapeutic compound to a human or animal comprising mixing an effective amount of said therapeutic compound with a pharmaceutically acceptable viscous compound and providing said mixture to said human or animal for oral consumption.

2. A method according to claim 1 wherein said viscous compound is spread on a serving structure such that full oral consumption by said human or animal requires repeated licking of said mixture.

3. A method according to claim 1 wherein said viscous compound is spread inside a serving structure such that full oral consumption by said human or animal requires repeated licking of said mixture.

4. A method according to claim 1 wherein said animal is a dog.

5. A method according to claim 1 wherein said animal is a cat.

6. A method according to claim 1 wherein the viscous compound is flavored to appeal to a human or animal.

7. A method according to claim 1 wherein said therapeutic compound contains allergenic extracts.

8. A method according to claim 1 wherein the viscous compound additionally contains any of a prebiotic agent and a probiotic agent.

9. A method according to claim 1 wherein said viscous carrier is paste.

10. A method according to claim 1 wherein said viscous carrier is a gel.

11. A method according to claim 1 wherein said composition is in the form of an aqueous solution.

12. A composition comprising:
a. one or more antigenic extracts; and
b. a viscous compound.

13. A composition according to claim 11 wherein said viscous compound is a paste, gelatin, gum, or cellulosic compound.

14. A composition according to claim 11 wherein said viscous compound is peanut butter.

15. A composition according to claim 11 wherein said composition additionally comprises a prebiotic.

16. A composition according to claim 11 wherein said composition additionally comprises a probiotic.
